(19) United States
(12) Patent Application Publication
(10) Pub. No.: US 2026/0298858 A1
(43) Pub. Date: Oct. 1, 2026

(54) ULTRASONIC ELECTROMAGNETIC SENSOR FOR MULTIPHASE CUT MEASUREMENTS AND ASSOCIATED METHODS

(71) Applicant: PERM Inc., Calgary (CA)

(72) Inventors: Milad ARABLOO, Calgary (CA); Mojtaba HASIRI, Calgary (CA); Apostolos KANTZAS, Calgary (CA); Saeid KHASI, Calgary (CA); Ilia KUZNETCOV, Calgary (CA)

(21) Appl. No.: 19/560,489

(22) Filed: Mar. 9, 2026

Related U.S. Application Data
(60) Provisional application No. 63/781,069, filed on Mar. 31, 2025.

(51) Int. Cl. G01N 27/22; G01F 1/58; G01F 1/667; G01F 1/74; G01N 33/28

(52) U.S. Cl. CPC G01N 27/221; G01F 1/584; G01F 1/667; G01F 1/74; G01N 33/2823

(57) ABSTRACT

The present disclosure relates to a system for multiphase fraction analysis of a sample. The system has an electromagnetic emitter configured to emit an electromagnetic signal into the sample and an electromagnetic sensor for measuring the electromagnetic signal after it has interacted with the sample. The system also has an ultrasonic emitter configured to emit an ultrasonic signal into the sample and an ultrasonic sensor configured to measure the ultrasonic signal after it has interacted with the sample. A controller determines the phases of the sample based on the measured electromagnetic and ultrasonic signals.

### RELATED APPLICATION

The present application claims 35 U.S.C. 119 priority from U.S. Provisional Application Ser. No. 63/781,069 filed on Mar. 31, 2025, entitled, “Ultrasonic Electromagnetic Sensor for Multiphase Cut Measurements and Associated Methods”, which is hereby incorporated by reference in its entirety.

### TECHNICAL FIELD

The invention relates to multiphase flow metering and real-time fluid characterization, and in particular to inline measurement of water, oil, and gas volume fractions in industrial pipelines.

### BACKGROUND

Traditional multiphase flow meters—particularly those using Nuclear Magnetic Resonance (NMR) technology—are accurate but extremely expensive, complex, and impractical for widespread industrial deployment, especially in cost-sensitive or remote applications.

NMR-based sensors can also be bulky, and require specialized infrastructure and safety protocols.

Alternative methods include gamma-ray densitometers, capacitance sensors, and Coriolis meters, but each comes with limitations in accuracy, safety, or usability, especially in dynamic or heterogeneous flow conditions.

Despite advances, many existing technologies lack portability or ease of integration in existing pipeline systems; are cost-prohibitive, limiting their use to high-end applications, struggle with real-time, accurate measurement of dynamic three-phase mixtures; and may involve radiation hazards or complex calibration requirements.

U.S. provisional application No. 62/958,091, U.S. provisional application No. 63/067,725, and U.S. application Ser. No. 17/130,184 entitled “Methods and Apparatus for Determining the Spatial Distribution of Materials Using Electromagnetic Radiation” relate to apparatus for determining the spatial distribution of materials within a sample using electromagnetic waves. The apparatus probes a sample with electromagnetic waves, measures the response, and determines the spatial configuration based on the measured response. U.S. provisional application No. 62/958,091, U.S. provisional application No. 63/067,725, and U.S. application Ser. No. 17/130,184 are hereby incorporated by reference in their entirety.

### SUMMARY

In accordance with the present disclosure, there is provided a system for multiphase fraction analysis of a sample, the system comprising:
an electromagnetic sensing assembly comprising:
   an electromagnetic emitter configured to emit an electromagnetic signal into the sample; and
   an electromagnetic sensor configured to measure the electromagnetic signal after the electromagnetic signal has interacted with the sample;
an ultrasonic sensing assembly comprising:
   an ultrasonic emitter configured to emit an ultrasonic signal into the sample; and
   an ultrasonic sensor configured to measure the ultrasonic signal after the ultrasonic signal has interacted with through the sample; and
a controller configured to determine the phases of the sample based on the measured electromagnetic and ultrasonic signals.

The sample may comprise multiple fluids. The sample may comprise multiple phases. The sample may comprise multiple states of matter (e.g., liquid and gas).

The system may comprise a conduit, and wherein the electromagnetic and ultrasonic emitters are configured to emit the electromagnetic and ultrasonic signals into the sample as the sample flows through the conduit.

The system may comprise a sample container. The sample container may comprise one or more of: a vessel, a conduit, a pipe and/or a tank.

The ultrasonic sensor may be configured to measure the time of flight of the ultrasonic signal through the sample. The controller is configured to determine the fraction of gas within the sample based on the measured time of flight.

The ultrasonic signal may comprise multiple frequencies.

The electromagnetic sensor may be configured to measure the permittivity of the flowing medium.

The electromagnetic emitter may be configured to emit electromagnetic waves into a sample along an emitter axis.

The emitter axis may be aligned with the vertical.

The emitter axis may be transverse to a direction of flow of the sample.

The electromagnetic sensor may be positioned to receive electromagnetic waves after the electromagnetic signal has interacted with the sample by at least one of: reflection and transmission.

The electromagnetic assembly may be configured to detect a frequency response of the measured electromagnetic signal. The frequency response may relate to how the sample interacts with the electromagnetic signal across a range of frequencies.

The electromagnetic sensing assembly may comprise an electromagnetic reflection sensor and/or an electromagnetic transmission sensor.

The electromagnetic sensing assembly may comprise a waveguide comprising multiple aligned conductors configured to support Transverse Electro-Magnetic modes within the waveguide.

The waveguide may be aligned vertically. The waveguide may extend substantially across the full dimension of the sample container.

Each conductor may be an elongate conductor. Using elongate conductors may help fluid flow through and within the waveguide. It will be appreciated that in multi-phase mixtures, it is important that all of the phases can freely move through and within the waveguide to allow an accurate measurement of the various levels.

The system may comprise one or more of: multiple electromagnetic sensing assemblies; and multiple ultrasonic sensing assemblies. Multiple sensing assemblies may be used to determine turbulence or differences in levels at different regions of the sample container.

The ultrasonic and electromagnetic sensing assemblies may be positioned in different positions within the sample container. The ultrasonic and electromagnetic sensing assemblies may be positioned adjacent to each other within the sample container. The controller may take into account the flow rate of the contents within the sample container to correlate measurements of the ultrasonic and electromagnetic sensing assemblies. For example, in a conduit, if the ultrasonic and electromagnetic sensing assemblies are spaced apart by 10 cm, and the flow rate in the conduit is 1 m/s, the controller may offset the reading of the upstream sensing assembly by 0.1 second to allow a direct comparison. This may be particularly important for rapidly changing flows.

The controller may be configured to control the ultrasonic and electromagnetic emitters. E.g., the controller may control the emitters to emit the signals at particular times and/or time intervals. The controller may control the frequency components of the signals themselves.

The ultrasonic emitter may be configured to emit the ultrasonic signal into the sample and towards the ultrasonic sensor. The ultrasonic sensing assembly may be configured to operate in a transmission mode.

The ultrasonic emitter and ultrasonic sensor may be positioned adjacent to each other. The ultrasonic emitter may be configured to emit the ultrasonic signal into the sample away from the ultrasonic sensor. The ultrasonic sensing assembly may be configured to operate in a reflection mode.

According to a further aspect, there is provided a method for multiphase fraction analysis of a sample, the method comprising:
emitting an electromagnetic signal into the sample;
measuring the electromagnetic signal after it has interacted with the sample;
emitting an ultrasonic signal into the sample;
measuring the ultrasonic signal after it has interacted with through the sample; and
determining the phases of the sample based on the measured electromagnetic and ultrasonic signals.

The signals may interact with the sample by passing through the sample.

The sample may comprise at least two of: gas, oil and water.

The ultrasonic signal may comprise signals with frequencies greater than 20 kilohertz. The ultrasonic signals span frequencies of between 20 kHz and 100 KHz.

The system may comprise an electromagnetic reflection sensor (e.g., for measuring reflected signals) and an electromagnetic transmission sensor (e.g., for measuring transmitted signals).

The range of electromagnetic frequencies may span frequencies between 2 MHz and 6 GHz.

The system may use ultrasonic and electromagnetic (EM) sensors in a single integrated system for real-time, inline measurement of water, oil, and gas volume fractions.

Ultrasonic time-of-flight (ToF) measurements may be used for determining a gas fraction in a multiphase sample (e.g., in a stationary or flowing sample).

Electromagnetic conductivity measurements may be used for quantifying water content within the fluid mixture.

The system may use a mathematical algorithm and method for calculating oil fraction based on the relationship.

The sensor may be configured and placed within the pipe system to optimize or improve detection sensitivity and minimize signal interference.

The system may use a data processing and interpretation method that combines input from both sensor types to accurately and dynamically characterize three-phase flows.

The system may use non-invasive or minimally intrusive hardware design for deployment in industrial pipelines without disrupting flow or requiring complex retrofitting.

The system may provide a compact system architecture that provides a practical alternative to traditional NMR-based multiphase meters.

According to a further aspect of the present disclosure, there is provided a non-transitory medium storing a computer program, the computer program configured to, when run on a computer:
process received data corresponding to a measured electromagnetic signal after it has interacted with a sample;
process received data corresponding to a measured ultrasonic signal after it has interacted with the sample; and
determine the phases of the sample based on the measured electromagnetic and ultrasonic signals.

Also disclosed is a software interface and firmware used for data acquisition, real-time monitoring, and flow visualization.

In the context of this disclosure, a phase is a region of material that is chemically consistent and physically distinct. A phase may be mechanically separable from other phases. A sample may comprise multiple liquid phases (e.g., immiscible phases such as oil and water).

In the context of this disclosure, a fraction of a phase may be the proportion of that phase within the whole sample. It may be expressed as a volumetric ratio or percentage.

The present technology integrates ultrasonic (US) and electromagnetic (EM) sensing techniques for accurate and cost-effective analysis.

The system may comprise a temperature sensor. The system may comprise a pressure sensor. Integration with temperature and pressure sensors may allow for temperature and/or pressure compensation and improved accuracy.

The system may use machine learning algorithms to enhance phase fraction prediction under varying flow regimes.

The system may use multi-frequency ultrasonic pulses to improve resolution.

The system may have swappable or modular sensor heads for different pipe materials or diameters.

The system may comprise a wireless communication module for remote monitoring and IoT (Internet of Things) integration.

The apparatus may comprise a temperature sensor (or a thermometer) within the core holder. The thermometer may comprise fiber optic thread.

The controller may comprise a processor and memory. The memory may store computer program code. The processor may comprise, for example, a central processing unit, a microprocessor, an application-specific integrated circuit or ASIC or a multicore processor. The memory may comprise, for example, flash memory, a hard-drive, volatile memory. The computer program may be stored on a non-transitory medium such as a CD. The computer program may be configured, when run on a computer, to implement methods and processes disclosed herein. Also, the controller may be implemented, for example, using a WiFi module of the Raspberry Pi™ (or other alternative of the microcomputer) and the running server client on this computer. The output may be displayed on the online page of the browser and be accessed from anywhere via internet.

In the context of a VNA network, scattering parameters (or S-parameters) describe the electrical behavior of electrical networks when undergoing various electrical stimuli. For example, S-parameters can be measured to characterize the electrical properties or performance of a radio frequency (RF) component. In this case, the radio frequency (RF) component comprises the sample.

In the S-parameter nomenclature, Syx, the second number (x) represents the originating connector, while the first number is the destination connector (y). That is:
S11 indicates that connector 1 is where the signal originates and where the response is detected and corresponds to reflection;
S21 indicates that connector 1 is where the signal originates and connector 2 where the response is detected and corresponds to transmission;
S22 indicates that connector 2 is where the signal originates and where the response is detected and corresponds to reflection (but from the opposite connector to S11); and
S12 indicates that connector 2 is where the signal originates and connector 1 where the response is detected and corresponds to transmission (but in the opposite direction to S21).

### BRIEF DESCRIPTION OF THE DRAWINGS

In the Detailed Description section below, one or more embodiments of the present technology are described in relation to the attached figures. These embodiments are intended to provide a better understanding of the invention, how the invention may be put into practice, and to demonstrate some of the advantages of the invention. The drawings are not necessarily to scale, emphasis instead being placed upon illustrating the principles of various embodiments of the invention. Similar reference numerals indicate similar components.

FIG. 1a is a schematic of a coaxial line configuration showing signal transmission from a generator to a load.

FIG. 1b is a visualization of the transverse electric and magnetic fields in a coaxial transmission line.

FIG. 2 is a cross-sectional view of a first embodiment of the system within a pipe conduit.

FIG. 3a-e are respectively an end view, a side view, a top view and two perspective views of a further embodiment of the system within a pipe conduit.

### DETAILED DESCRIPTION

### Introduction

A goal is to develop a more affordable, compact, and practical inline sensing system that can measure water, oil, and gas fractions with reliable accuracy.

The technology integrates two sensing technologies—ultrasonic measurement (e.g., using pulse echo measurements) and electromagnetic (EM) permittivity sensing—to determine the volumetric fractions of gas, water, and oil in a flowing multiphase stream.

The ultrasonic component may comprise a pulse transmitter and receiver mounted in line with a pipeline. It measures the time of flight (ToF) of ultrasonic pulses through the fluid mixture. The speed of sound varies significantly between gas, oil, and water, allowing this measurement to estimate the gas fraction.

The EM sensor may be positioned to measure the permittivity of the flowing medium/sample. Since water is conductive while oil and gas are not, this measurement is used to calculate the water fraction.

The oil fraction may then be determined by material balance.

In this way, the components of a three-phase mixture of oil, water and gas can be determined using a combination of two sensing assemblies.

Advantages may include one or more of the following:
Non-invasive or minimally intrusive design
Lower cost compared to NMR or gamma-ray-based systems
No radiation hazards, unlike gamma densitometers
Real-time inline monitoring capability
Compact and scalable for different pipe sizes

The combination of sensors allows for accurate, real-time, and safe multiphase flow measurement, particularly valuable for oil and gas production, separation units, and enhanced oil recovery (EOR) operations.

This technology may be used in a wide range of applications including:
Oil and Gas pipelines
Chemical and petrochemical industries
Water treatment (e.g., oil-water separation monitoring)
Multiphase reactors in process engineering
Environmental monitoring (e.g., detecting oil contamination in water systems)
Food processing (e.g., dairy or beverage production where multiphase flow monitoring is needed)

Various aspects of the invention will now be described with reference to the figures. For the purposes of illustration, components depicted in the figures are not necessarily drawn to scale. Instead, emphasis is placed on highlighting the various contributions of the components to the functionality of various aspects of the invention. A number of possible alternative features are introduced during the course of this description. It is to be understood that, according to the knowledge and judgment of persons skilled in the art, such alternative features may be substituted in various combinations to arrive at different embodiments of the present invention.

### Electromagnetic Parameters

To achieve the research objectives, a specific type of coaxial line probe was designed, developed, and commissioned. This EM sensor is cost-effective, easy to install in operational environments, and highly versatile for applications ranging from oil and gas monitoring to environmental material characterization. The probe operates over a wide range of frequencies, extending up to the GHz range. The inner and outer conductors are interchangeable, allowing customization for various applications. A larger outer diameter increases the sensing volume, capturing average properties over a broader region, whereas smaller configurations provide localized measurements.

The fabrication process involved precision machining of conductive components, while certain structural elements were sourced from commercially available components. The accuracy of the probe was validated against reference materials, such as pure dielectric samples, ensuring its reliability for experimental use. This custom-designed probe provides a practical and scalable solution for dielectric property measurements across diverse research and industrial applications. By integrating this probe into the experimental setup, this study enhances the ability to characterize complex multiphase systems, contributing to improved understanding and optimization of fluid behavior in energy and environmental applications.

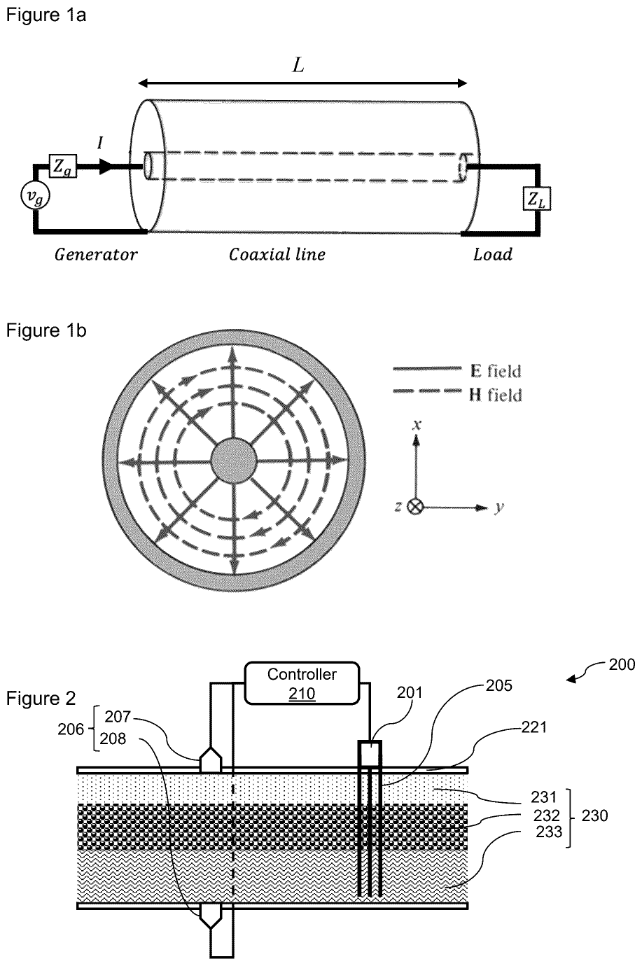

FIG. 1a is a schematic of a coaxial line configuration showing signal transmission from a generator to a load. FIG. 1b is a visualization of the transverse electric and magnetic fields in a coaxial transmission line.

Considering a transmission line of length L, characterized by propagation constant (Y) and characteristic impedance (Zc), connected to a load Z_{L} as shown in FIGS. 1a and 1b:
V₀⁺ and V₀⁻ are the forward and reflected voltage wave amplitudes.
γ=α+jβ is the complex propagation constant, with:
   α as the attenuation constant (Np/m),
   β as the phase constant (rad/m).
Zc is the characteristic impedance of the transmission line.

Looking into the line, the generator sees the line with the load as an input impedance Zin. The input impedance Zin is derived using voltage and current wave equations. The general form of these equations is:

$$V ( z ) = {V}_{c}^{+} {e}^{- γ z} + {V}_{c}^{-} {e}^{γ z} \qquad ( 1 )$$

$$I ( z ) = \frac{{V}_{0}^{+}}{{Z}_{c}} {e}^{- γ z} - \frac{{V}_{0}^{-}}{{Z}_{c}} {e}^{γ z} \qquad ( 2 )$$

Applying boundary conditions at z=−L, the input impedance Zin at the generator end is given by the generalized transmission line equation:

$${Z}_{i n} = {Z}_{c} \frac{{Z}_{L} + {Z}_{c} tan ( γ L )}{{Z}_{c} + {Z}_{L} tan ( γ L )} \qquad ( 3 )$$

For an open-circuited line (Z_{L}=∞) the above equation becomes:

$${Z}_{i n} = {Z}_{c} \frac{1}{tanh ( γ L )} = {Z}_{c} coth ( γ L ) \qquad ( 4 )$$

The reflection coefficient at the transition from cable to probe, as measured by the VNA, is related to Z_{T} and Z₀ via the following equation:

$$Γ = \frac{{Z}_{T} - {Z}_{0}}{{Z}_{T} + {Z}_{0}} \qquad ( 5 )$$

In which Z₀=50 ohm (which is the internal impedance of the VNA). Re-arrangement gives:

$${Z}_{i n} = {Z}_{0} \frac{1 + Γ}{1 - Γ} \qquad ( 6 )$$

$${Z}_{i n} = {Z}_{c} coth ( γ L ) \qquad ( 7 )$$

By equating two equations:

$${Z}_{i n} = {Z}_{0} \frac{1 + Γ}{1 - Γ} = {Z}_{c} coth ( γ L ) \qquad ( 8 )$$

$${Z}_{0} \frac{1 + Γ}{1 - Γ} = \sqrt{\frac{R + j ω L}{G + j ω C}} coth ( \sqrt{( R + j ω L ) ( G + j ω C )} L ) \qquad ( 9 )$$

$${Z}_{0} \frac{1 + Γ}{1 - Γ} = \sqrt{\frac{\frac{{R}_{S}}{2 π} ( \frac{1}{a} + \frac{1}{b} ) + j ω \frac{{μ}_{0}}{2 π} ln ( \frac{b}{a} )}{\frac{2 π ω {ε}_{0} {ε}_{r}^{″}}{ln ( \frac{b}{a} )} + j ω \frac{2 π {ε}_{0} {ε}_{r}^{′}}{ln ( \frac{b}{a} )}}} coth ( \sqrt{( \frac{{R}_{S}}{2 π} ( \frac{1}{a} + \frac{1}{b} ) + j ω \frac{{μ}_{0}}{2 π} ln ( \frac{b}{a} ) ) ( \frac{2 π ω {ε}_{0} {ε}_{r}^{″}}{ln ( \frac{b}{a} )} + j ω \frac{2 π {ε}_{0} {ε}_{r}^{′}}{ln ( \frac{b}{a} )} )} L ) \qquad ( 10 )$$

Mathematically, total impedance is related to the scattering parameter (or S-parameter) S11 as:

$${Z}_{i n} = {Z}_{0} \frac{1 + {S}_{11}}{1 - {S}_{11}} \qquad ( 11 )$$

where Z₀=50Ω (the characteristic impedance of the coaxial cable).

$${Z}_{0} \frac{1 + {S}_{11}}{1 - {S}_{11}} = \sqrt{\frac{\frac{{R}_{S}}{2 π} ( \frac{1}{a} + \frac{1}{b} ) + j ω \frac{{μ}_{0}}{2 π} ln ( \frac{b}{a} )}{\frac{2 π ω {ε}_{0} {ε}_{r}^{″}}{ln ( \frac{b}{a} )} + j ω \frac{2 {πε}_{0} {ε}_{r}^{′}}{ln ( \frac{b}{a} )}}} coth ( \sqrt{( \frac{{R}_{S}}{2 π} ( \frac{1}{a} + \frac{1}{b} ) + j ω \frac{{μ}_{0}}{2 π} ln ( \frac{b}{a} ) ) ( \frac{2 π ω {ε}_{0} {ε}_{r}^{″}}{ln ( \frac{b}{a} )} + j ω \frac{2 π {ε}_{0} {ε}_{r}^{′}}{ln ( \frac{b}{a} )} )} L ) \qquad ( 12 )$$

As noted above, In the S-parameter nomenclature, S11 indicates that connector 1 is where the signal originates and where the response is detected and corresponds to reflection.

In the previous equation, ¿, appears in a non-linear, transcendental form inside both the square root and the cotangent function, along with a term of square root multiplied by the cotangent term. This makes the analytical solution to isolate εᵣ explicitly very difficult. Therefore, alternative solutions could be solving for εᵣ using optimization packages or applying an approximation instead of the cotangent term by using the Taylor series to simplify the equation.

### Ultrasonic Parameters

The speed of sound can be calculated from Time-of-Flight measurements as follows. Given known distance L between transmitter and receiver:

$$C = \frac{L}{ToF} \qquad ( 13 )$$

Where:
c is the effective speed of sound in the multiphase mixture;
L is the distance between transducers (meters); and
ToF is Time of flight (seconds).

The Gas Volume Fraction can be calculated from Effective Speed of Sound using Wood's Equation for a two-phase (gas-liquid) mixture:

$$\frac{1}{{c}^{2}} = \frac{{∅}_{g}}{{c}_{g}^{2}} + \frac{{∅}_{w}}{{c}_{w}^{2}} + \frac{{∅}_{o}}{{c}_{o}^{2}} \qquad ( 14 )$$

$${∅}_{9} + {∅}_{o} + {∅}_{w} = 1 \qquad ( 15 )$$

$$\frac{1}{{c}^{2}} = \frac{{∅}_{g}}{{c}_{g}^{2}} + \frac{{∅}_{w}}{{c}_{w}^{2}} + \frac{1 - {∅}_{g} - {∅}_{w}}{{c}_{o}^{2}} \qquad ( 16 )$$

$${∅}_{g} = \frac{\frac{1}{{c}^{2}} - \frac{1}{{c}_{o}^{2}} - {∅}_{w} ( \frac{1}{{c}_{o w}^{2}} - \frac{1}{{c}_{o}^{2}} )}{\frac{1}{{c}_{g}^{2}} - \frac{1}{{c}_{o}^{2}}} \qquad ( 17 )$$

Where:
c: measured sound speed (via ToF)
c_{g}: sound speed in gas (e.g., ~343 m/s)
c_{w}: sound speed in water (e.g., ~1500 m/s)
cₒ: sound speed in oil (e.g., ~1400 m/s)
φ_{w}: water fraction (from EM sensor).
φ_{g}: gas fraction (what we solve)
φₒ: oil fraction

### First Embodiment

FIG. 2 shows an embodiment of the system 200 in conjunction with a pipe conduit 221. In this case, the pipe is carrying a three-phase sample 230 comprising a phase of gas 231, a first liquid phase of oil 232, and a second liquid phase of water 233.

In this embodiment, the system comprises:
an electromagnetic sensing assembly 201 comprising:
   an electromagnetic emitter configured to emit an electromagnetic signal into the sample; and
   an electromagnetic sensor configured to measure the electromagnetic signal after it has passed through the sample;
an ultrasonic sensing assembly 206 comprising:
   an ultrasonic emitter 207 configured to emit an ultrasonic signal into the sample;
   an ultrasonic sensor 208 configured to measure the ultrasonic signal after it has passed through the sample; and
a controller 210 configured to determine a fraction of multiple phases of the sample based on the measured electromagnetic and ultrasonic signals.

In this case, the electromagnetic and ultrasonic emitters are configured to emit the electromagnetic and ultrasonic signals into the sample as if flows through the conduit, and the controller is configured to determine the fraction of the multiple phases of the sample. In this way, the system may be able to monitor the phases within the sample in real time. This can be important in use cases where the phase fractions may change unexpectedly over time. For example, in oil production, the oil fraction, water fraction and gas fraction may change over time.

The electromagnetic sensing assembly in this case comprises an electromagnetic emitter and an electromagnetic sensor, which are configured to measure the permittivity of the flowing medium.

As described above, the permittivity determination may be used to determine the fraction of a conductive phase (e.g., water) within a sample consisting of the conductive phase and other non-conductive phases. For example, the electromagnetic assembly may be used by the controller, as in this case, to determine the fraction of the water phase within a sample consisting of water and oil and/or gas.

The electromagnetic emitter is configured to emit electromagnetic waves into a sample along an emitter axis which, in this case, is aligned with the vertical and transverse to a direction of flow of the sample.

The electromagnetic sensor is configured to detect the electromagnetic waves emitted by the electromagnetic emitter. The electromagnetic sensor (or sensors) is positioned to receive electromagnetic waves after the electromagnetic waves have interacted with the sample by at least one of: reflection and transmission, and wherein the detector is configured to detect a frequency response of the received electromagnetic waves.

In this embodiment, the electromagnetic sensing assembly comprises an electromagnetic reflection sensor. Other embodiments may comprise an electromagnetic transmission sensor instead of, or in addition to, an electromagnetic reflection sensor.

In this embodiment, the system comprises a waveguide 205 comprising multiple aligned conductors configured to support Transverse Electro-Magnetic modes within the waveguide.

In this embodiment, the ultrasonic sensor is configured to measure the time of flight of the ultrasonic signal, and wherein the controller is configured to determine the fraction of gas within the sample based on the measured time of flight.

In this embodiment, the ultrasonic sensing assembly is configured to emit an ultrasonic signal vertically through the sample from the ultrasonic emitter 207 to the ultrasonic sensor 208. Based on the time of flight through the sample, and using known speeds of sound within the various phases, the fractional ratio of the gas portion can be determined by the controller.

With the water fraction and the gas fraction determined, the controller may then determine the remaining oil fraction. In this way, the fraction of all three phases may be determined using the two sensing assemblies.

It will be appreciated that, in other embodiments, the ultrasonic sensing assembly may be configured to detect reflections of the ultrasonic signal from one or more phase boundaries within the sample.

In this embodiment, the ultrasonic signal comprises multiple frequencies. This may help increase accuracy, e.g., by using the frequency-dependent speeds of sound for the various phases.

### Second Embodiment

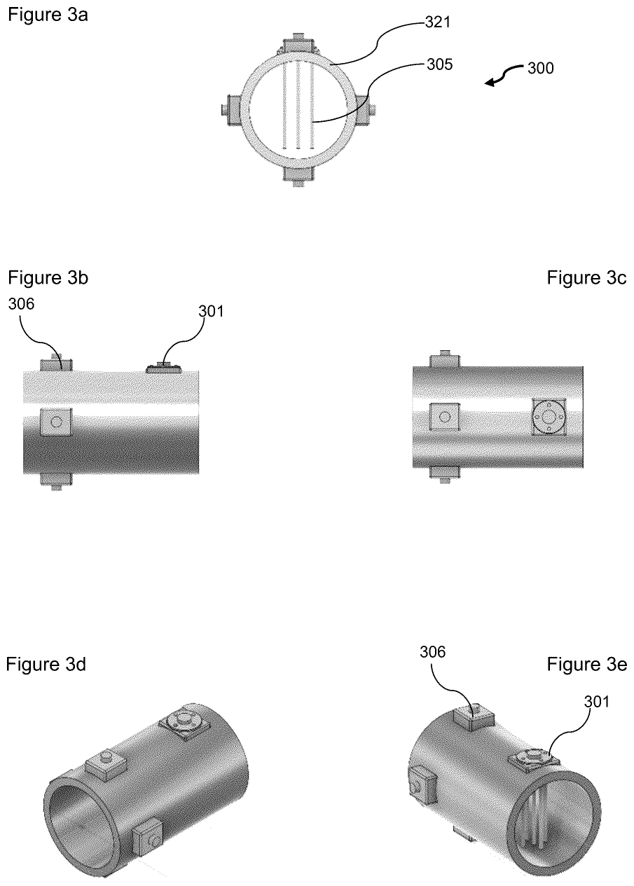

FIGS. 3a-e are various views of a further system 300 for measuring the fractions of various phases within a multiphase mixture. In this case, the controller is not shown.

As in the previous embodiment, the system is being used in conjunction with a pipe conduit 321 and comprises an electromagnetic sensing assembly 301 with a waveguide and an ultrasonic sensing assembly 306.

As shown in FIG. 3e, the waveguide comprises five electrodes including a central electrode defining a central waveguide axis, and four aligned electrodes arranged equidistantly around the central waveguide axis. It will be appreciated that other waveguide configurations may be used in other embodiments.

### Testing

Initial tests conducted in a controlled lab setup demonstrated accurate correlation between ultrasonic ToF and known gas fractions.

Electromagnetic measurements were validated using different mixtures of oil and water to estimate water content.

A proof-of-concept prototype was developed and tested with known multiphase mixtures to verify sensor performance.

Although the present invention has been described and illustrated with respect to preferred embodiments and preferred uses thereof, it is not to be so limited since modifications and changes can be made therein which are within the full, intended scope of the invention as understood by those skilled in the art.

1. A system for multiphase fraction analysis of a sample, the system comprising:
an electromagnetic sensing assembly comprising:
an electromagnetic emitter configured to emit an electromagnetic signal into the sample; and
an electromagnetic sensor configured to measure the electromagnetic signal after the electromagnetic signal has interacted with the sample;
an ultrasonic sensing assembly comprising:
an ultrasonic emitter configured to emit an ultrasonic signal into the sample; and
an ultrasonic sensor configured to measure the ultrasonic signal after the ultrasonic signal has interacted with through the sample; and
a controller configured to determine the phases of the sample based on the measured electromagnetic and ultrasonic signals.

2. The system according to claim 1, wherein the system comprises a conduit, and wherein the electromagnetic and ultrasonic emitters are configured to emit the electromagnetic and ultrasonic signals into the sample as the sample flows through the conduit.

3. The system according to claim 1, wherein the ultrasonic sensor is configured to measure the time of flight of the ultrasonic signal through the sample, and wherein the controller is configured to determine the fraction of gas within the sample based on the measured time of flight.

4. The system according to claim 1, wherein the ultrasonic signal comprises multiple frequencies.

5. The system according to claim 1, wherein the electromagnetic sensor is configured to measure the permittivity of the flowing medium.

6. The system according to claim 1, wherein the electromagnetic emitter is configured to emit electromagnetic waves into a sample along an emitter axis.

7. The system according to claim 6, wherein the emitter axis is aligned with the vertical.

8. The system according to claim 6, wherein the emitter axis is transverse to a direction of flow of the sample.

9. The system according to claim 6, wherein the electromagnetic sensor is positioned to receive electromagnetic waves after the electromagnetic signal has interacted with the sample by at least one of: reflection and transmission.

10. The system according to claim 6, wherein the electromagnetic assembly is configured to detect a frequency response of the measured electromagnetic signal.

11. The system according to claim 6, wherein the electromagnetic sensing assembly comprises an electromagnetic reflection sensor and an electromagnetic transmission sensor.

12. The system according to claim 1, wherein the electromagnetic sensing assembly comprises a waveguide comprising multiple aligned conductors configured to support Transverse Electro-Magnetic modes within the waveguide.

13. The system according to claim 12, wherein the waveguide is aligned vertically.

14. The system according to claim 1, wherein the system comprises one or more of: multiple electromagnetic sensing assemblies; and multiple ultrasonic sensing assemblies.

15. The system according to claim 1, wherein the controller is configured to control the ultrasonic and electromagnetic emitters.

16. The system according to claim 1, wherein the ultrasonic emitter is configured to emit the ultrasonic signal into the sample and towards the ultrasonic sensor.

17. The system according to claim 1, wherein the electromagnetic sensing assembly is configured to determine a fraction of a conductive phase within a sample consisting of a conductive phase and one or more non-conductive phases.

18. A method for multiphase fraction analysis of a sample, the method comprising:
emitting an electromagnetic signal into the sample;
measuring the electromagnetic signal after it has interacted with the sample;
emitting an ultrasonic signal into the sample;
measuring the ultrasonic signal after it has interacted with the sample; and
determining the phases of the sample based on the measured electromagnetic and ultrasonic signals.

19. The method of claim 18, wherein the sample comprises at least two of: gas, oil and water.

20. A non-transitory medium storing a computer program, the computer program configured to, when run on a computer:
process received data corresponding to a measured electromagnetic signal after it has interacted with a sample;
process received data corresponding to a measured ultrasonic signal after it has interacted with the sample; and
determine the phases of the sample based on the measured electromagnetic and ultrasonic signals.
